(19) United States
(12) Patent Application Publication
(10) Pub. No.: US 2014/0071401 A1
(43) Pub. Date: Mar. 13, 2014

(54) Retinoscope

(71) Applicant: Zumax Medical Co., Ltd, Jiangsu (CN)

(72) Inventors: Jilong Wang, Jiangsu (CN); Jin He, Jiangsu (CN); Shunguo Shen, Jiangsu (CN); Xiangdong Li, Jiangsu (CN); Weizhong Zhou, Jiangsu (CN); Xiaohu Liu, Jiangsu (CN)

(73) Assignee: Zumax Medical Co., Ltd, Jiangsu (CN)

(21) Appl. No.: 13/921,405

(22) Filed: Jun. 19, 2013

(30) Foreign Application Priority Data
Sep. 9, 2012 (CN) 201210320252.3

(51) Int. Cl. A61B 3/103

(52) U.S. Cl. CPC A61B 3/1035
USPC 351/214

(57) ABSTRACT

A retinoscope for detecting eye ametropia and astigmatism axes is disclosed in the invention, which comprises an illuminating and imaging system comprising an illumination light source, a projecting mirror and a half transparent and half reflecting mirror arranged sequentially along the direction of propagation of the illumination light, the respective optical axes of the projecting mirror and half transparent and half reflecting mirror are collinear with each other, and the reflecting plane of the half transparent and half reflecting mirror is at an angle of 45° relative to the illumination optical axis, wherein a condensing lens column and a retinoscopy diaphragm are disposed sequentially along the illumination optical axis between the illumination light source and the projecting mirror, the optical axis of the condensing lens column is coincident with the illumination optical axis, and the plane of the retinoscopy diaphragm is perpendicular to the illumination optical axis.

### FIELD OF THE INVENTION

The invention relates to photoelectric lighting and display technique, and more particularly to a retinoscope.

### BACKGROUND OF THE INVENTION

Retinoscopes are devices for detecting eye ametropia and astigmatism axis, in which optical systems utilizing filaments for illumination and imaging are provided. When a retinoscope is used for detecting the dioptric situation of an eyeball objectively, generally, inside of the eyeball is illuminated by the retinoscope, and light is reflected back from the retina, the dioptric situation will change when the reflected light passes through the eyeball, thus, the dioptric situation of the eyeball can be determined by checking the change of the reflected light.

Generally, retinoscopes can be divided into two types according to the types of the used light, namely, streak retinoscopes and spot retinoscopes.

Currently, the streak retinoscopes are usually used for clinical optometry. The strip light utilized in an existing retinoscope is provided by filaments of a filament lamp with high brightness, the filaments achieve imaging and illumination via a projecting mirror. The above retinoscope mainly comprises a filament lamp, a projecting mirror, and a half transparent and half reflecting mirror, wherein the filament lamp serves as a light source of linear filaments and the image of filaments is formed and reflected into an eyeball of people by the projecting mirror and the half transparent and half reflecting mirror respectively. A checker performs optometry by observing changes in the imaging of the filaments and adjusting simultaneously the distance between the projecting mirror and the lamp bulb, to change the projecting beams to divergent beams or convergent beams. However, there are some disadvantages in the above retinoscopes of prior art, such as, the strip image of the filaments is not clear and uniform enough, light energy of the light source is not utilized effectively, and lifetime of the filament lamp is short.

### SUMMARY OF THE INVENTION

One object of the present invention is to provide a retinoscope comprising an illuminating and imaging system by which light energy can be utilized effectively.

For the above purpose, the following technical solution is utilized in the invention:

A retinoscope for detecting eye ametropia and astigmatism axis, comprises an illuminating and imaging system having an illumination light source, a projecting mirror and a half transparent and half reflecting mirror arranged sequentially along the direction of propagation of the illumination light. The respective optical axes of the projecting mirror and the half transparent and half reflecting mirror are collinear with each other, while the reflecting plane of the half transparent and half reflecting mirror is at an angle of 45° relative to the illumination optical axis. A condensing lens column and a retinoscopy diaphragm are disposed sequentially along the illumination optical axis between the illumination light source and the projecting mirror, the optical axis of the condensing lens column being coincident with the illumination optical axis, and the plane of the retinoscopy diaphragm being perpendicular to the illumination optical axis.

Preferably, the condensing lens column comprises single lens or multiple lenses.

More preferably, the retinoscopy diaphragm is a strip diaphragm or a spot diaphragm.

Preferably, the retinoscopy diaphragm is disposed rotatably about the illumination optical axis.

More preferably, the projecting mirror is disposed movably along the illumination optical axis relative to the retinoscopy diaphragm.

Still more preferably, the illumination light source is a filament lamp, or a xenon lamp, or a halogen lamp, or a LED lamp.

Preferably, the illumination light source is a white light source or colored light source.

More preferably, the half transparent and half reflecting mirror is replaceable by a reflecting mirror with holes.

Still more preferably, the half transparent and half reflecting mirror is replaceable by a reflecting mirror plated with a hollow reflecting film.

As compared with the prior art, the invention has following advantages due to use of the above technical solution:

The retinoscope of the present invention is provided with a condensing lens column between the illumination light source and retinoscopy diaphragm, this causes that the light energy can be used effectively, and the image of strip light or spot light from the retinoscopy diaphragm formed on the eyeball to be detected has a more clear and uniform boundary. Furthermore, the retinoscope with such illuminating and imaging system has compact configuration. Additionally, better color vision can be achieved if a colored light source is utilized, such as a red or green light source, and thus the imaging can be distinguished by a user.

### BRIEF DESCRIPTION OF DRAWINGS

FIG. 1 is a schematic drawing showing an illuminating and imaging system of the invention.

FIG. 2 is a schematic drawing showing a retinoscopy diaphragm of the invention, wherein the retinoscopy diaphragm is a strip diaphragm.

FIG. 3 is a schematic drawing showing a retinoscopy diaphragm of the invention, wherein the retinoscopy diaphragm is a spot diaphragm.

FIG. 4 is a schematic drawing showing a retinoscope of the invention.

wherein: 1, an illumination light source; 2, a condensing lens column; 3, a retinoscopy diaphragm; 4, a projecting mirror; 5. a half transparent and half reflecting mirror; 6, a first protective glass sheet; 7, a second protective glass sheet; 8, an observation eye; 9, an extinction sheet set; OO₁, an illumination optical axis; O₂O₃, an observation optical axis; P, a first image plane; M, an eyeball.

### DETAILED DESCRIPTION OF THE INVENTION

The invention will be further illustrated with reference to accompanying drawings and embodiments, however, it should be appreciated that the embodiments are given only by way of example and are not considered as limitation to the invention.

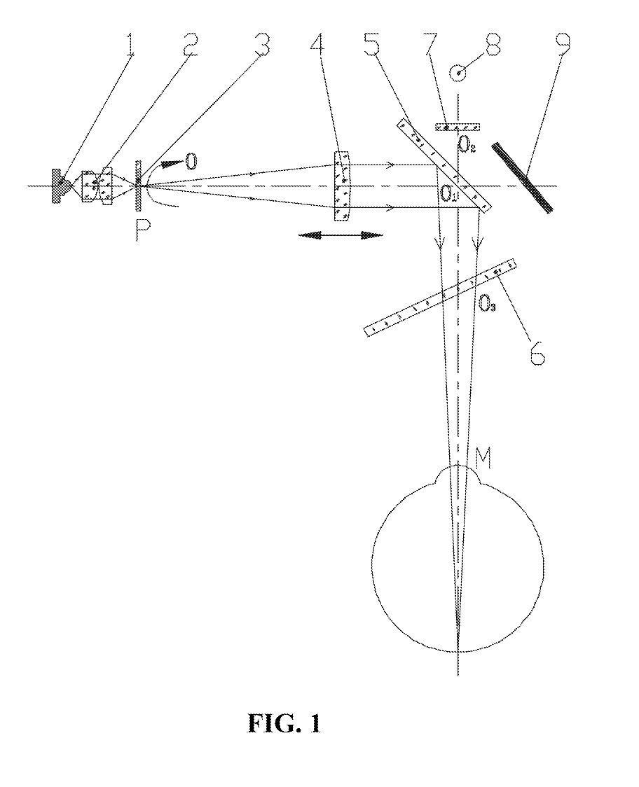

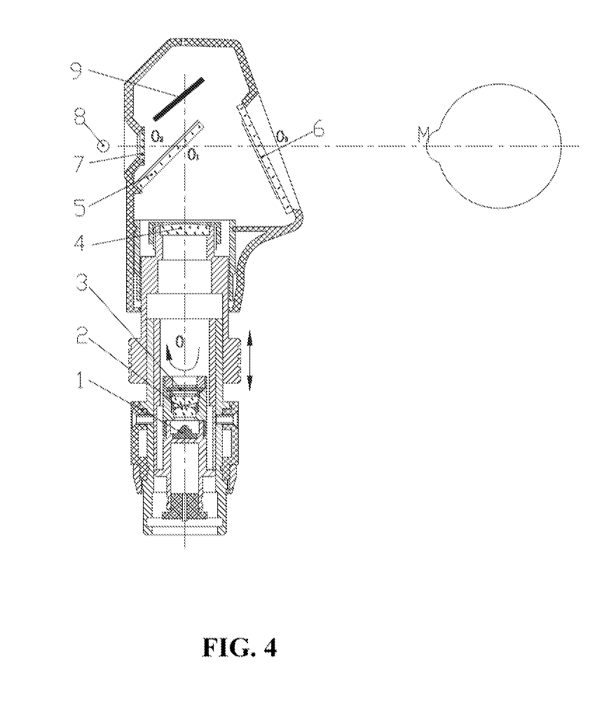

Referring to FIG. 1 and FIG. 4, the retinoscope of the embodiment is provided with an illuminating and imaging system, the illuminating and imaging system comprises an illumination light source 1, a condensing lens column 2, a retinoscopy diaphragm 3, a projecting mirror 4, a half transparent and half reflecting mirror 5 and an extinction sheet set 9 which are arranged sequentially along the direction of propagation of the illumination light. A first protective glass sheet 6 and a second protective glass sheet 7 are disposed at both sides of the half reflecting mirror 5 along an optical axis O₂O₃ of the reflected light beams from the half reflecting mirror 5, wherein the optical axis O₂O₃ is an observation optical axis perpendicular to the illumination optical axis OO₁.

The condensing lens column 2 may comprise single lens or multiple lenses.

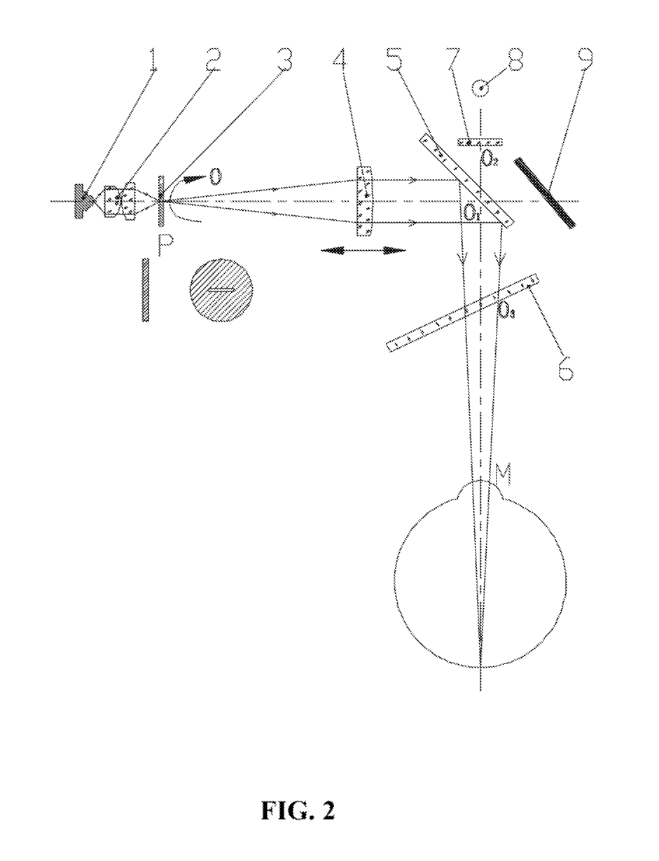

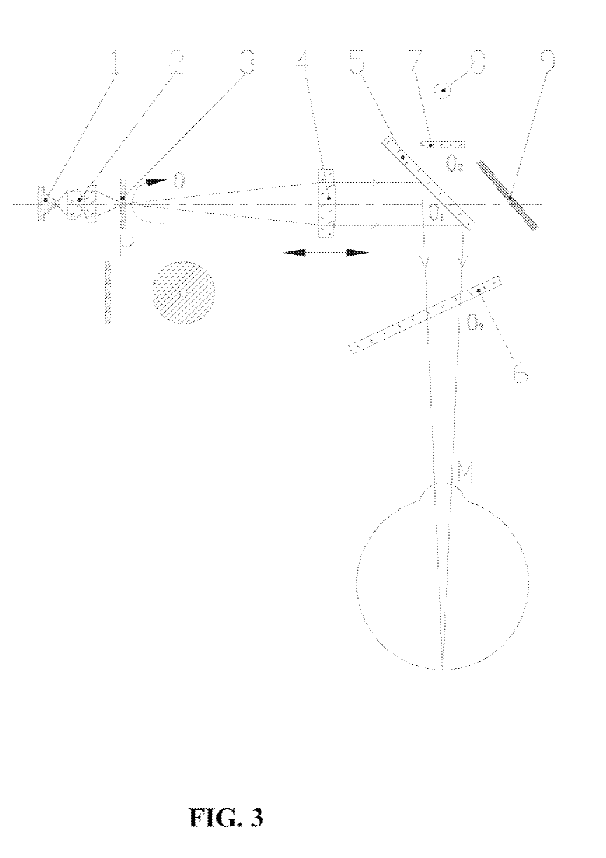

The retinoscopy diaphragm 3 is a strip retinoscopy or a spot retinoscopy, as shown in FIG. 2 and FIG. 3.

An extinction sheet set 9 is arranged behind the half transparent and half reflecting mirror 5, that is to say, if the front of the half transparent and half reflecting mirror 5 faces to the incident light beams, then the extinction sheet set 9 is disposed at back side of the half transparent and half reflecting mirror 5.

The illumination light source 1 may be a filament lamp, or a xenon lamp, or a halogen lamp, or a LED lamp, which can be a white light source or colored light source or the like.

The half transparent and half reflecting mirror 5 can be replaced by a reflecting mirror with holes or a reflecting mirror plated with a hollow reflecting film.

The illumination light beams pass through the condensing lens column 2 to form a first image plane P on which a retinoscopy diaphragm 3 is disposed. The plane of the side of the retinoscopy diaphragm 3 facing to the light beams is perpendicular to the illumination optical axis OO₁ and intersects the illumination optical axis OO₁ at O, wherein the retinoscopy diaphragm 3 can be rotated about the illumination optical axis OO₁. Furthermore, the projecting mirror 4 can be moved along the illumination optical axis OO₁ relative to the retinoscopy diaphragm 3, and the reflecting plane of the half transparent and half reflecting mirror 5 is at an angel of 45° relative to the illumination optical axis. The strip/spot light formed by the retinoscopy diaphragm 3 is projected onto the half transparent and half reflecting mirror 5 by the projecting mirror 4 and further reflected by the half transparent and half reflecting mirror 5, then the reflected light passes through a first protective glass sheet 6 to image on a eyeball M to be detected, any changes of the strip/spot light on the eyeball M to be detected are observed by an observation eye 8 of an observer via a second protective glass sheet 7, the half transparent and half reflecting mirror 5, and a first protective glass sheet 6. Additionally, for detecting refractive errors of a eye, the projecting mirror 4 is moved along the illumination optical axis OO₁ to adjust the size and clearness of the imaging of the retinoscopy diaphragm 3, and for detecting astigmatism axes on the eyeball M, the retinoscopy diaphragm 3 is rotated about the illumination optical axis OO₁, so that the imaging of the strip/spot light rotates about the observation optical axis O₂O₃.

The working principle of the retinoscope of the invention is described in more detail as follows:

With reference to the embodiment shown in FIG. 2, the light emitted from the illumination light source 1 passes through the condensing lens column 2 to project into the retinoscopy diaphragm 3 disposed on the image plane P, then the light beams from the retinoscopy diaphragm 3 are projected onto the half transparent and half reflecting mirror 5 by the projecting mirror 4, the direction of the light beams is changed after reflection of the half transparent and half reflecting mirror 5, and subsequently the reflected light beams pass through the first protective glass sheet 6 to shine on the eyeball M to be detected for detection. The observation eye 8 of the observer observes the eyeball to be detected via the second protective glass sheet 7, the half transparent and half reflecting mirror 5 and the first protective glass sheet 6. The size and clearness of the imaging of retinoscopy diaphragm 3 on the eyeball can be adjusted by moving the projecting mirror 4 along the illumination optical axis OO₁ to detect refractive errors of eyes. Furthermore, in order to detect astigmatic axes on the eyeball M, the retinoscopy diaphragm 3 is rotated about the illumination optical axis OO₁, so that the imaging of the retinoscopy diaphragm 3 rotates about the observation optical axis O₂O₃.

Furthermore, better color vision can be achieved if a colored light source is utilized, such as a red or green light source, thus, the imaging can be distinguished more easily by a user.

It should be noted that the above embodiments are only intended to illustrate the technical concept and features of the invention, instead of limitation. The object of the embodiments is in that a person skilled in the art can appreciate and further implement the invention. Any variations or equal replacements of the technical scheme of the invention are covered within the scope of the invention, without departing from the spirit and scope of the invention.

What is claimed is:

1. A retinoscope for detecting eye ametropia and astigmatic axis, which is provided with an illuminating and imaging system comprising an illumination light source, a projecting mirror and a half transparent and half reflecting mirror arranged sequentially along an illumination optical axis in such a way that the respective optical axes of the projecting mirror and the half transparent and half reflecting mirror are collinear with each other, and the reflecting plane of the half transparent and half reflecting mirror is at an angle of 45° relative to the illumination optical axis, wherein a condensing lens column and a retinoscopy diaphragm are disposed sequentially along the illumination optical axis between the illumination light source and the projecting mirror such that the optical axis of the condensing lens column is coincident with the illumination optical axis, and the plane of the retinoscopy diaphragm is perpendicular to the illumination optical axis.

2. The retinoscope as claimed in claim 1, wherein the condensing lens column comprises a single lens or a plurality of lenses.

3. The retinoscope as claimed in claim 1, wherein the retinoscopy diaphragm is a strip diaphragm or a spot diaphragm.

4. The retinoscope as claimed in claim 1, wherein the retinoscopy diaphragm is rotatable about the illumination optical axis.

5. The retinoscope as claimed in claim 1, wherein the projecting mirror is movable along the illumination optical axis relative to the retinoscopy diaphragm.

6. The retinoscope as claimed in claim 1, wherein the illumination light source is selected from the group consisting of a filament lamp, a xenon lamp, a halogen lamp, and a LED lamp.

7. The retinoscope as claimed in claim 1, wherein the illumination light source is a white light source or a colored light source.

8. The retinoscope as claimed in claim 1, wherein the half transparent and half reflecting mirror can be replaced by a reflecting mirror with holes.

9. The retinoscope as claimed in claim 1, wherein the half transparent and half reflecting mirror can be replaced by a reflecting mirror plated with a hollow reflecting film.
